(19) United States
(12) Patent Application Publication
(10) Pub. No.: US 2026/0215770 A1
(43) Pub. Date: Jul. 30, 2026

(54) ORAL CIRCUMFERENTIAL RESPIRATORY MONITORING DEVICE

(71) Applicants: Alain Jaccard, Sainte-Croix (CH); SedateUK Ltd, London (GB)

(72) Inventors: Alain Jaccard, Sainte-Croix (CH); Andreia Trigo, London (GB); Martin Lees, London (GB); Craig Cook, London (GB); Philippe Margairaz, Neuchâtel (CH)

(73) Assignee: SedateUK Ltd, London (GB)

(21) Appl. No.: 19/130,529

(22) PCT Filed: Nov. 21, 2023
(86) PCT No.: PCT/US2023/080628

§ 371 Date: May 15, 2025

Related U.S. Application Data
(60) Provisional application No. 63/426,816, filed on Nov. 21, 2022.

(51) Int. Cl. A61B 17/02; A61B 5/00; A61B 5/083; A61B 5/097; A61M 16/06

(52) U.S. Cl. CPC A61B 17/0293; A61B 5/0836; A61B 5/097; A61B 5/682; A61M 16/0672; A61B 2505/05

(57) ABSTRACT

A device is provided that enables the continuous and unobstructed sampling of CO₂ expired by a patient and administration of O₂ to said patient while at the same time allowing surgery or instrumentation or access of the oral cavity and related areas of said patient to proceed unobstructed.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND

It is known that when a dentist treats a patient, for example, for periodontal disease, during treatment the mouth needs to be kept open in order to provide optimal surgical access. Consequently, different oral circumferential holding devices are known.

### SUMMARY OF THE INVENTION

It is an object of the invention, to develop the device to provide optimal surgical access, deliver supplemental oxygen and continuously monitor inspired and expired gases as claimed in the appended claims. This object is met by an oral circumferential holding device adapted to hold the oral cavity of a patient open so as to allow breathing by the patient through the oral cavity and/or through the patient's nostril or nostrils, the holding device including an expandable internal flange and an expandable external flange, the external flange having an outer edge, the outer edge including an edge portion adapted to be placed adjacent the nostrils of a patient, and sampling tubing, for collecting air exhaled by the patient through the nose and/or the mouth, said sampling tubing connectable to a capnography machine to enable the uninterrupted and unobstructed sampling of CO₂ expired by a patient and, optionally, to enable the administration of O₂ to said patient, while at the same time allowing surgery or instrumentation of the oral cavity and related areas of said patient to proceed unobstructed.

This task is also solved by an oral and nasal capnography system adapted to facilitate monitoring of a patient, the system including the oral circumferential holding device of any one of the above claims, and further including a capnography machine which is adapted to analyze levels of CO₂ in the patient.

A further development of the sampling tubing includes nasal cannulae arrangeable in or adjacent to the nostrils of the patient. The nasal cannulae could be integrated into the edge portion of the external flange. In this way, a solution for analyzing the exhaled air has been found.

The sampling tubing may comprise a mouth exhaust manifold, preferably integrated in an edge of the oral circumferential holding device, the mouth exhaust manifold comprising holes, slots and/or channels being interspaced along a circumference of the internal flange, the holes, slots and/or channels preferably being adapted to be disposed along the oral/buccal cavity of a patient, adjacent to oral/buccal mucosa, when the device is installed.

Alternatively or additionally to the nasal cannulae, the sampling tubing may comprise the mouth exhaust manifold for analyzing the exhaled air.

In a further development the oral circumferential holding device may comprise additional tubing for O₂ nasal and mouth supply.

In a further development the oral circumferential holding device may include external connections for connecting the additional tubing to O₂ supply and connections for connecting the sampling tubing to the capnography machine. The advantage to this solution is an easy way to lead the exhaled air to the capnography machine.

The flanges may be biased to expand so as to help retract a patient's lip and/or cheek.

The external flange may be contoured so as to fit in the full external circumference of the lips and oral cavity.

An edge of the internal flange and/or an edge of the external flange is made of firm, reinforcing yet flexible and bendable plastic type tubing, connected by an elastic non-reinforced material.

A further development of the oral circumferential holding device includes a Y-connector adapted to join at least two branches of the sampling tubing into one tube for connection to a capnography machine. Therefore the air exhaled by the patient, can be easily analyzed.

The internal flange may be internally contoured so as to fit in the full internal circumference of oral cavity and mucosa.

A further development of the oral and nasal capnography system may include an oxygen supply and monitoring system for administering oxygen to the patient.

A further development of the oral and nasal capnography system comprises an “OPTRAGATE”™-like device and, a capnography sampling line/device, for example, a “MICROSTREAM”™ or a similar device.

An object of the invention is to provide a device that enables the uninterrupted and unobstructed sampling of CO₂ expired by a patient and administration of O₂ to said patient, while at the same time allowing surgery or instrumentation of the oral cavity and related areas of said patient to proceed unobstructed.

### BRIEF DESCRIPTION OF THE DRAWINGS

The integrated drawings represent, by way of example, different embodiments of the subject of the invention.

FIG. 1 is a schematic, front view of the device of the invention, worn by a patient.

FIG. 2 is a schematic view of the device of the invention.

FIG. 3A is a cross section of the device of FIG. 2, taken along line A-A.

FIG. 3B is a cross section of the device of FIG. 2, taken along line B-B.

FIG. 3C is a cross section of the device of FIG. 2, taken along line C-C.

FIG. 4 is another schematic, rear view of the device of the invention.

FIG. 5 is a sectional view of the collection ring of the invention.

FIG. 6A is an alternate schematic front view of the device of the invention, connected to Oxygen and CO₂ monitoring.

FIG. 6B is an alternate schematic rear view of the device of the invention, connected to Oxygen and CO₂ monitoring.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as it is exemplary in nature, serving to describe the best mode of the invention known to the inventors as of the filing date hereof. Consequently, changes may be made in the arrangement and/or function of any of the elements described in the exemplary embodiments disclosed herein without departing from the spirit and scope of the invention.

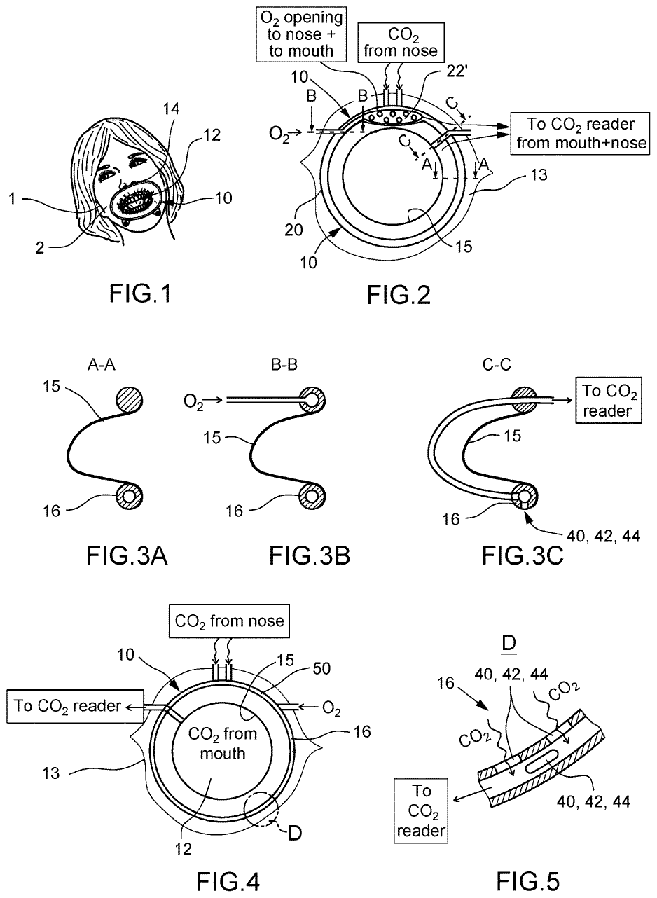

Referring now to FIG. 1, an oral circumferential holding device 10 adapted to hold the oral cavity 12 of a patient 1 open against the lips 13 so as to retract the cheeks 2 and so allow breathing by the patient through the oral cavity and/or through the patient's nostril or nostrils 14.

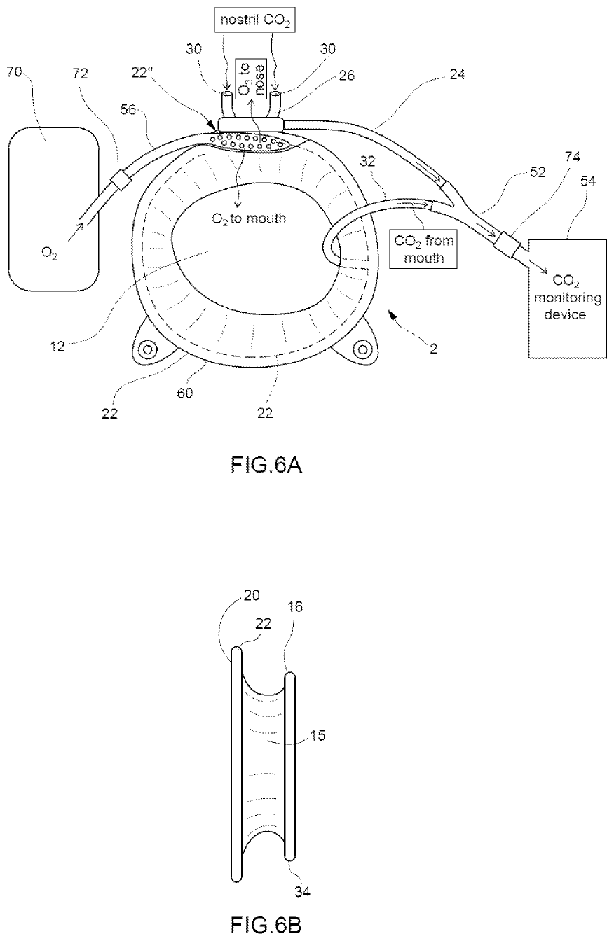

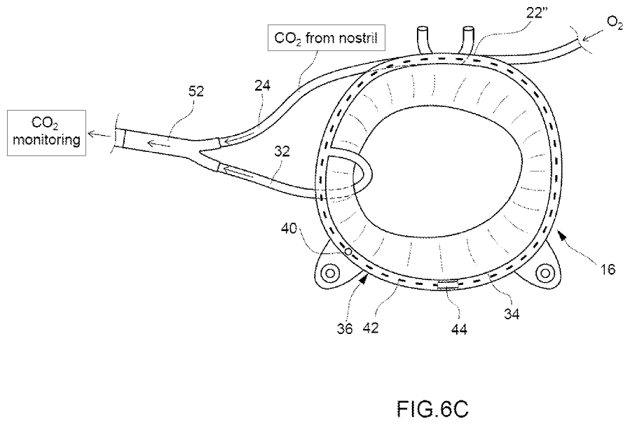

Referring now to FIG. 2, FIGS. 3A to 3C, and FIG. 4, also FIGS. 6A and 6B, the holding device 10 includes semi-rigid, flexible or expandable internal and external flanges 16, 20 connected by a diaphragm 15, the external flange 20 having an outer edge 22, including a perforated edge portion 22″ adapted to be placed adjacent the nostrils 14 of a patient 1 and connected to an oxygen source 70 via tubing 56 for administering oxygen. Sampling tubing 26 comprises nasal cannulae 30 orientated in or below the nostrils of the patient 1 and is connected via tubing 24 and 26 to a CO₂ or capnography device 54 as is sampling tubing 32 preferably integrated in or connected to the edge 34 of the flange 16 of the holding device 10 which acts as a manifold 36 (which may be a second tube adjacent the internal flange 16 itself, the tube(s) from which it is made having perforations or slots to enable CO₂ sensing) in which holes 40, slots 42, or channels 44 (see FIG. 5) are interspaced along a circumference 50 of the internal flange 16. Optionally, each flange 16, 20 may be made expandable using two flexible tubes, one of which is sized to pass inside the other, fixed together—glued—at one end in a short overlapping area wherein at the free end, the other smaller diameter end passes into the larger diameter end, advantageously biased to expand by a long expansion spring is inserted in the larger diameter tube so as to act against the end of the smaller diameter tube or a thin spring or flexible rod is inserted. The sampling nostril tubing 26 and the internal sampling tube 32 optionally integrated into the internal flange 16 are connectable to a capnography machine 54 (using the Y-connector 52 and the connection tube 74) to enable the uninterrupted and unobstructed sampling of CO₂ expired by a patient and, optionally, to enable the administration of O₂ through an oxygen tube 56 to said patient, while at the same time allowing surgery or instrumentation of the oral cavity and related areas of said patient to proceed in an unobstructed manner.

The device 10 of the invention may also integrate capnography sampling via a CO₂ monitoring device 54 into an oral circumferential device which acts as a lip and cheek retractor device (similar to an “OPTRAGATE”™ device by Ivoclar Vivadent AG, a latex-free lip and cheek retractor which is flexible and elastic so as to be comfortable to wear and assist patients to keep their mouth open) that is highly flexible and elastic in all directions of movement for patient comfort and assists in keeping the patient's mouth open, while at the same time sampling patients inspired/inhaled and expired/exhaled air, without obstructing the surgical procedure. Monitoring of CO₂ or capnography is performed through integration of the use of standard capnography sampling lines (such as the Medtronic “MICROSTREAM”™ device).

The device 10 of the invention optionally includes:
Oral circumferential device (items 15, 16, 20), and with width-spanning internal and external aspects of the oral cavity via the expandable or flexible flanges 16, 20;
Superior top edge 22 of flange 20 externally contoured to the full external circumference of the lips 13 and mouth 12;
Inferior bottom (internal) flange 16 internally contoured to the full internal circumference of oral cavity and mucosa;
Both flanges 16, 20 made of firm, reinforcing yet flexible and bendable plastic type tubing, connected by an elastic non-reinforced material, with a low hardness in the range of 30 A to 50 A; such as present in an off-the-shelf “OPTRAGATE”™ device;
CO₂ capnography performed via a CO₂ monitoring device connected to the internal flange 16 and/or to nasal cannulae 30 via sampling tubing 24 integrated into edges of the oral circumferential device
Superior top edge: Superior sampler via nasal cannulae orientated in or below nostrils (as with/similar to normal nasal cannulae)
Inferior bottom edge: Inferior sampler via holes (or slots) interspaced at regular intervals the full circumference of oral cavity edge
O₂ nasal and mouth supply via oriented specific holes
Additional tubing to connect inferior tubing to Y-connector
Y-connector joining superior and inferior tubing into one tube for connection to capnography machine.
External connections to O₂ supply and capnography machine

A device according to the invention may be made by combining a modified “OPTRAGATE”™-like device or similar, and combining with a “MICROSTREAM”™ device or similar capnography sampling line/device. Additional tubing is added to ensure CO₂ monitoring from inside the mouth.

The invention can be summarized as including at least one or a portion of one of the following feature sets:
1. An oral circumferential holding device (10) adapted to hold the oral cavity (12) of a patient open so as to allow breathing by the patient through the oral cavity and/or through the patient's nostril or nostrils (14) while at the same time allowing surgery or instrumentation of the oral cavity and related areas of said patient to proceed unobstructed, the holding device including:
   an expandable internal flange (16) and an expandable external flange (20), the external flange (20) having an outer edge (22), the outer edge (22) including an edge portion (22″) adapted to be placed adjacent the nostrils (14) of a patient, and
   sampling tubing (24, 26, 32), for collecting air exhaled by the nose and/or by the mouth of the patient, said sampling tubing (24, 32) connectable to a capnography machine (54) to enable the uninterrupted and unobstructed sampling of CO₂ expired by said patient.
2. An oral circumferential holding device (10) according to feature set 1, wherein the sampling tubing (26) comprise nasal cannulae (30) arrangeable in or adjacent to the nostrils of the patient.
3. The oral circumferential holding device (10) according to feature set 2, wherein the nasal cannulae (30) are integrated into the edge portion (22″) of the external flange (20).
4. An oral circumferential holding device (10) according to one of the preceding feature sets, wherein the sampling tubing comprises a mouth exhaust manifold (36), preferably integrated in an edge (34) of the oral circumferential holding device (10), the mouth exhaust manifold (36) comprising holes (40), slots (42) and/or channels (44) being interspaced along a circumference (50) of the internal flange (16).
5. The oral circumferential holding device (10) according to one of the preceding feature sets comprising additional tubing (56) for O₂ nasal and mouth supply.
6. The oral circumferential holding device (10) according to the above feature set, including external connections (72) for connecting the additional tubing (56) to O₂ supply and connections (74) for connecting the sampling tubing (24, 26) to the capnography machine (54).
7. The oral circumferential holding device (10) according to one of the preceding feature sets wherein the flanges (16, 20) are biased to expand so as to help retract a patient's lip and/or cheek.
8. The oral circumferential holding device (10) according to one of the preceding feature sets, wherein the external flange (20) is contoured so as to fit in the full external circumference of the lips and oral cavity (12).
9. The oral circumferential holding device (10) according to one of the preceding feature sets, wherein an edge of the internal flange (16) and/or an edge of the external flange (20) is made of firm, reinforcing yet flexible and bendable plastic type tubing (60), connected by an elastic non-reinforced material.
10. The oral circumferential holding device (10) according to one of the preceding feature sets, including a Y-connector (52) adapted to join at least two branches of the sampling tubing (24, 26, 32, 66) into one tube for connection to the capnography machine (54).
11. The oral circumferential holding device (10) according to one of the preceding feature sets, wherein the internal flange (16) is internally contoured so as to fit in the full internal circumference of oral cavity (12) and mucosa.
12. An oral and nasal capnography system (100) adapted to facilitate monitoring of a patient, the system including the oral circumferential holding device (10) of any one of the above feature sets, and further including a capnography machine (54) which is adapted to analyze levels of CO₂ in the patient.
13. The oral and nasal capnography system (100) of the above feature set, including an oxygen supply and monitoring system (70) for administering oxygen to the patient.
14. An oral circumferential holding device (10) according to feature set 1, wherein the sampling tubing (26) is at least partially integrated in the external flange (20).
15. The oral and nasal capnography system (100) according to one of the feature sets 12 to 13, comprising a capnography sampling line/device (54), preferably a “MICROSTREAM”™ or similar device.

In an advantage, a device 10 is provided that allows reliable expired CO₂ monitoring for a patient during procedures involving or requiring or accessing the oral cavity, while the patient is under sedation or anesthesia.

In a further advantage, a device is provided that enables the sampling of CO₂ expired by a patient and administration of O₂ to said patient while at the same time allowing surgery or instrumentation of the oral cavity and related areas of said patient to proceed unobstructed.

It should be appreciated that the implementations shown and herein described are representative of the invention and its best mode and are not intended to limit the scope of the present invention in any way.

As will be appreciated by skilled artisans, the present invention may be embodied as a system, a device, or a method.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited, but may also include other elements such as those described in the instant specification. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto and/or in the abstract. Such claims and/or abstract are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

1. An oral circumferential holding device adapted to hold the oral cavity of a patient open so as to allow breathing by the patient through the oral cavity and/or through the patient's nostril or nostrils while at the same time allowing surgery or instrumentation of the oral cavity and related areas of said patient to proceed unobstructed, the holding device including:
an expandable internal flange and an expandable external flange, the external flange having an outer edge, the outer edge including an edge portion adapted to be placed adjacent the nostrils of a patient, and
sampling tubing, for collecting air exhaled by the nose and/or by the mouth of the patient, said sampling tubing connectable to a capnography machine to enable the uninterrupted and unobstructed sampling of CO₂ expired by said patient.

2. An oral circumferential holding device according to claim 1, wherein the sampling tubing comprise nasal cannulae arrangeable in or adjacent to the nostrils of the patient.

3. The oral circumferential holding device according to claim 2, wherein the nasal cannulae are integrated into the edge portion of the external flange.

4. An oral circumferential holding device according to claim 1, wherein the sampling tubing comprises a mouth exhaust manifold, preferably integrated in an edge of the oral circumferential holding device, the mouth exhaust manifold comprising holes, slots and/or channels being interspaced along a circumference of the internal flange.

5. The oral circumferential holding device according to claim 1 comprising additional tubing for O₂ nasal and mouth supply.

6. The oral circumferential holding device according to claim 5, including external connections for connecting the additional tubing to O₂ supply and connections for connecting the sampling tubing to the capnography machine.

7. The oral circumferential holding device according to claim 1 wherein the internal flange and the external flange are biased to expand so as to help retract a patient's lip and/or cheek.

8. The oral circumferential holding device according to claim 1, wherein the external flange is contoured so as to fit in the full external circumference of the lips and oral cavity.

9. The oral circumferential holding device according to claim 1, wherein an edge of the internal flange and/or an edge of the external flange is made of firm, reinforcing yet flexible and bendable plastic type tubing, connected by an elastic nonreinforced material.

10. The oral circumferential holding device according to claim 1, including a Y-connector adapted to join at least two branches of the sampling tubing into one tube for connection to the capnography machine.

11. The oral circumferential holding device according to claim 1, wherein the internal flange is internally contoured so as to fit in the full internal circumference of oral cavity and mucosa.

12. An oral and nasal capnography system adapted to facilitate monitoring of a patient, the system including the oral circumferential holding device of claim 1, and further including a capnography machine which is adapted to analyze levels of CO₂ in the patient.

13. The oral and nasal capnography system of claim 12, including an oxygen supply and monitoring system for administering oxygen to the patient.

14. An oral circumferential holding device according to claim 1, wherein the sampling tubing is at least partially integrated in the external flange.

15. The oral and nasal capnography system according to claim 12, comprising a capnography sampling line/device.

16. (canceled)
